(19) United States
(12) Patent Application Publication
(10) Pub. No.: US 2025/0299828 A1
(43) Pub. Date: Sep. 25, 2025

(54) FRAMEWORK FOR INTEGRATION OF HEALTH INFORMATION IN MEDICINE

(71) Applicant: Siemens Healthineers AG, Forchheim (DE)

(72) Inventors: Shikha Chaganti, Princeton, NJ (US); Daphne Yu, Yardley, PA (US); Dorin Comaniciu, Princeton, NJ (US); Brian Da Silva Teixeira, Princeton, NJ (US); David Peicho, London (GB)

(21) Appl. No.: 18/863,478

(22) PCT Filed: May 18, 2022
(86) PCT No.: PCT/EP2022/063458

§ 371 Date: Nov. 6, 2024

(51) Int. Cl. G16H 50/30; G16H 10/60; G16H 15/00

(52) U.S. Cl. CPC G16H 50/30; G16H 10/60; G16H 15/00

(57) ABSTRACT

Health information is integrated (106) into a framework system (400). PGHD and clinical data are integrated (106) into a patient data model (240). The PGHD data as integrated may be compressed or otherwise processed (104) to reduce the volume and/or frequency of the data for greater ease in understanding the PGHD data. A user interface (220) for this data model (240) allows for access to both types of data (PGHD and clinical data) by a patient or a physician. Artificial intelligence may be used to further consolidate the data by providing one or more biomarkers (120) estimated from both types of data, allowing for patient and/or physician goal, treatment success, and/or adverse event monitoring.

### BACKGROUND

The present embodiments relate to integration of health information in medicine. Individual centric healthcare and wellness data has the potential for improving patient outcomes and empowering patients to take charge of their health. However, the clinical use of such patient-generated health data (PGHD) has largely not yet been adapted. The digital health data of an individual remains fragmented and resides in different silos, with interoperability still far from practical. While several digital health applications exist for patient engagement, these applications target a specific use case or a subset of patient-specific digital health data. PGHD gathering devices and applications are fast changing, and their data are not normalized across devices or manufacturers. The contexts from which the data is collected is also not managed or recorded, making it difficult to draw any meaningful conclusions.

For a healthcare provider, clinical data is generally available using various clinical systems. With increasing adoption of electronic health records in the clinical setting, a large amount of digital health datapoints are available for physicians today. Personal wellness devices and mobile applications outside the clinical setting result in even more information. Clinicians are not trained data analysts and are already overwhelmed with patient, provider, and lab information generated from clinics and hospitals.

### SUMMARY

By way of introduction, the preferred embodiments described below include methods, computer readable media, and systems for integration of health information into a framework system. PGHD and clinical data are integrated into a patient data model. The PGHD data as integrated may be compressed or otherwise processed to reduce the volume and/or frequency of the data for greater ease in understanding the PGHD data. A user interface for this data model allows for access to both types of data (PGHD and clinical data) by a patient or a physician. Artificial intelligence may be used to further consolidate the data by providing one or more biomarkers estimated from both types of data, allowing for patient and/or physician goal, treatment success, and/or adverse event monitoring.

In a first aspect, a method is provided for integration of health information into a framework system. Clinical data for a patient from a first source of medical healthcare is formatted into a first format for a patient data model of the framework system. Patient-generated health data from the patient is formatted into a second format for the patient data model of the framework system. The clinical data and patient-generated health data are integrated as formatted into the patient data model for the patient. The integration stores both the clinical data and the patient-generated health data together as the patient data model. An interface or interfaces to the patient data model are provided, including access to the clinical data and the patient-generated health data for both a patient and a physician. A machine-learned model generates a biomarker for the patient in response to input of at least some of both of the clinical data and the patient-generated health data from the patient data model. The biomarker is output.

In one embodiment, the clinical data is from a picture archiving and communications system (PACS), radiology information system, electronic health record, and/or laboratory information system for a medical practice or facility. The clinical data is formatted for integration into the data model, such as formatting pursuant to a standard for medical data sharing.

In another embodiment, the patient-generated health data is formatted for integration where the patient-generated health data is from a wearable sensor. The formatting from the wearable sensor may include compressing data from the wearable sensor provided at a first frequency to a less frequent representation. The formatting from the wearable sensor may include compressing the data from the wearable sensor to periodic event episodes based on event context of the data from the wearable sensor.

Various types of data are integrated. As one embodiment, the formatting of the patient-generated health data may be reformatting data from an application for gathering information input by the patient. For example, data from a wearable sensor is compressed. The clinical data being formatted for integration is a picture archiving and communications system (PACS) or radiology information system, an electronic health record, and laboratory information system for a medical practice or facility.

In yet another embodiment, the provided interface is display of an avatar of the patient with a characteristic of the avatar representing the biomarker. In a further approach, the avatar is displayed with a medical image from the clinical data. The medical image is selected for the displaying based on selection of a landmark or anatomical region of the avatar.

In various embodiments, the biomarkers are generated as a health score for overall health of the patient, a disease specific biomarker, a change leading to an adverse event, and/or a response to treatment and/or medication.

As one embodiment, the user interface provides a summary of the patient-generated health data to the physician.

In a second aspect, a system is provided for modeling patient data in a framework. A memory is configured to store clinical data from a database of a healthcare facility and to store patient-generated health data from a wearable sensor and/or application on a patient device. The clinical data and the patient-generated health data are stored in the framework common to both the clinical data and patient-generated health data. A processor is configured to generate a user interface for interacting with the framework by a patient including access to the clinical data. A display is configured to display part the user interface.

In one embodiment, the user interface includes a prediction application. The prediction application is configured to generate a health score for the patient based on both the clinical data and the patient-generated health data from the framework. As another embodiment, the processor is configured to summarize the patient-generated health data, and the processor is configured to generate the user interface for interacting with the framework by a physician. The summarization is available to the physician in the user interface. In yet another embodiment, the user interface includes an avatar of the patient and information from the clinical data based on selection of part of the avatar.

As a third aspect, a method is provided for integration of health information into a framework system. A patient data model of the framework system is populated through a connection to an application programming interface of a patient device of a patient. The patient data model is populated with a periodic aggregate summary of more frequent data collected by an application of the application programming interface. Clinical data for the patient from a healthcare facility is accessed. An artificial intelligence analyzes health of the patient based on the clinical data and the periodic aggregate summary. The health is presented to the patient on a user interface including an avatar of the patient. The avatar has a characteristic representing the health.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for integration of health information into a framework system;

FIG. 2 illustrates an example patient data model;

FIG. 3 is a table of types of healthcare information and corresponding sources;

FIG. 4 is a block diagram of a framework for generating a patient data model according to one embodiment;

FIG. 5 is a table of example events for a patient and corresponding relation with a patient data model;

FIGS. 6-8 illustrate use of a patient data model for goal monitoring, adverse event detection, and treatment follow-up, respectively;

FIG. 9 is a block diagram of one embodiment of a system for modeling patient data in a framework.

### DETAILED DESCRIPTION OF THE DRAWINGS AND PRESENTLY PREFERRED EMBODIMENTS

An avatar-based “My Digital Twin” framework is provided for lifelong or periodic health management. This health digital twin software brings in situ PGHD together with existing clinical data systems. Information for both patients and clinicians are enhanced via a patient data model (Digital Twin model). The framework is staged with variation points along the data flow from PGHD towards clinical use, such that the patient data model is capable of adapting to data transformation, normalization, and reduction to be performed along each step. Patient data samples are summarized into contextualized episodes and integrated with accessible clinical data to be stored in the common patient data model to be consumed by various Digital Twin engines, which provide insights for routine clinical use.

The framework provides a way to utilize data from patient and clinical applications in an integrated ecosystem of domain specific workflows. The framework also aims to connect the patient closer to their clinical data and vice versa. Patient friendly communication techniques for health information are incorporated within the communication capabilities of the framework, such as through the use of an avatar representation to communicate health data.

Due to new US Information Blocking rules and 21^{st} Century Cures Act, a lot of applications have risen that can connect to health information technology (IT) systems. The framework allows for diagnostics based on integrated data. The data from the clinical and patient sources are provided, as integrated, in a suitable manner to both clinicians and patients.

This framework provides a comprehensive patient-specific view of health and wellness and brings together clinical and in situ patient-generated health data. The integrated data within the framework is aggregated to a lifelong patient data model, with various applications and user interfaces tailored to communicate with the patient and clinical users in the loop. Adaptable variation points are built into the concept of the framework such that data normalization and reduction can be evolved over time to the pace of PGHD data sources. Consistent clinical presentation context and compatibility are, thus, provided to clinical traditions. Patient-specific health data including imaging, EHR, wearables and PGHD are comprehensively consolidated into the patient data model. The framework includes avatar generation (i.e., a personalized avatar in likeness of the patient to increase engagement with the application), patient empowerment (e.g., integrated data model facilitates quick and easy consultations), preventative care (e.g., provide actionable insights to improve and monitor health at the right time), virtual consultation and/or remote diagnosis (e.g., virtual portability of data improves access to diagnosis), remote patient monitoring (e.g., monitor and manage impact of therapy), and digital therapeutics (e.g., continuous management and behavioral therapies).

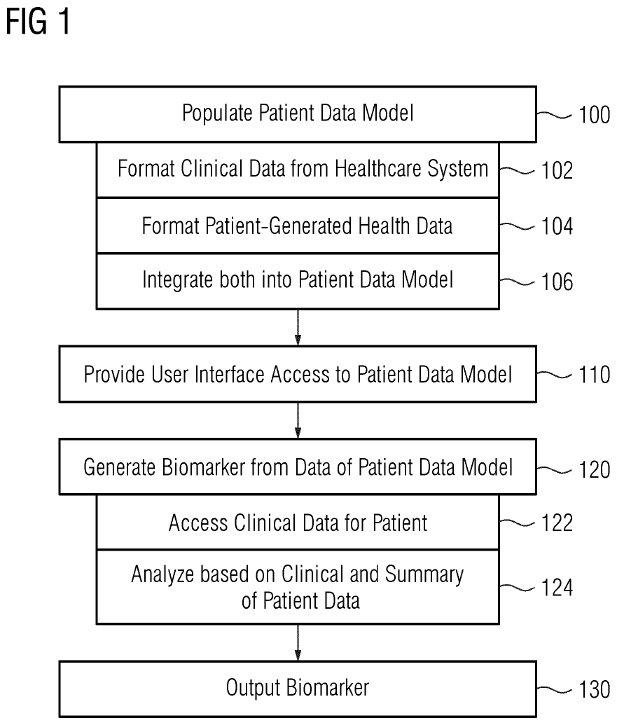

FIG. 1 shows a flow chart for one embodiment of a method for integration of health information into a framework system. Clinical data and PGHD are both integrated into the same patient data model. The patient and/or physician may then access both types of information. The patient-gathered data may be compressed into a summary for more concise information for a physician. The user interface for this framework may include an avatar for patient access.

The method is implemented by a server, workstation, computer, tablet computer, processor of mobile phone, combinations thereof, or another computer. For example, a server populates the patient data model. A memory stores the patient data model, and a computer of the patient or physician executes an application of the framework, which application accesses the patient data model for operation (e.g., generation and output of a biomarker). The same or different processor performs various of the acts. One or more memories local to or remote from the processor store the patient data model for use by the processor or processors. The output is to a display of a local processor or a remote display.

The method is implemented in the order shown or a different order. For example, acts 102, 104, and 106 are provided in other orders. As another example, act 110 may be provided before act 100.

Additional, different, or fewer acts may be performed. For example, acts 100-106 are not provided where the patient data model already exists. As another example, acts 120 and/or 130 are not performed. In another example, acts for gathering and/or using health information and/or acts for configuring or establishing the patient data model for a given patient are included.

In act 100, a computer populates a patient data model of the framework system. By accessing various sources using communications appropriate for those sources, health data is mined to populate the patient data model. The health data from various sources is gathered and provided as part of one patient data model, such as database formatted for housing the different types of data together as part of a framework for access by the patient and/or physician.

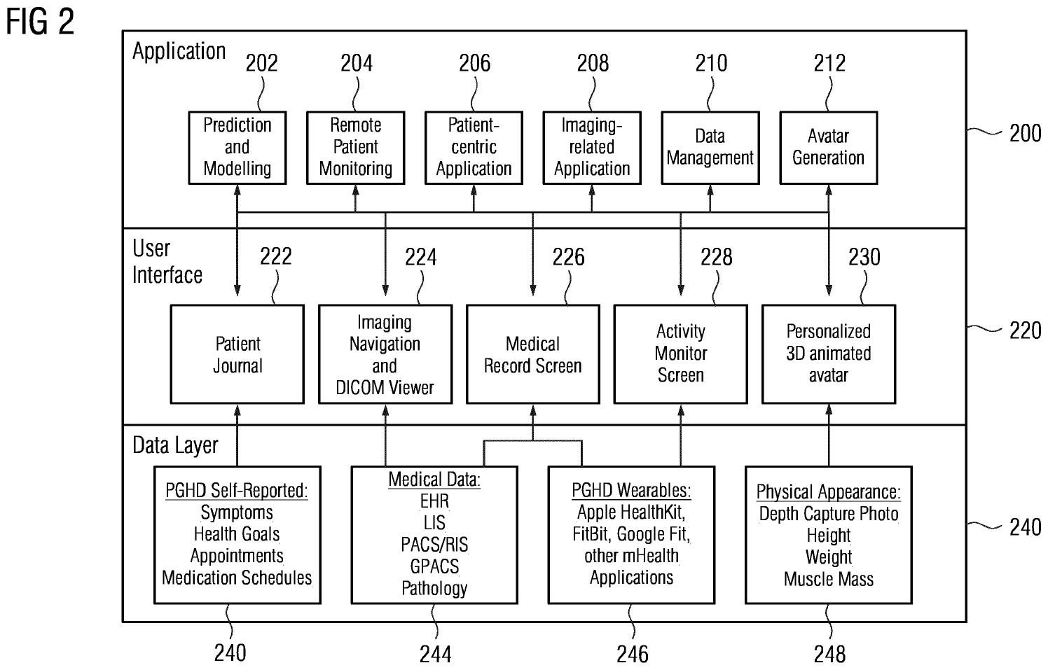

FIG. 2 shows the conceptual layers of the patient data model and corresponding flow for the proposed digital twin framework. Health and wellness data from multiple silos are integrated for presenting relevant healthcare information and insights with patient friendly visualization on a personal computing device. The framework includes one or more applications 200 using one or more user interfaces 220 based on health data in the patient data model 240 of the data layer. Additional, different, or fewer layers and corresponding computer instructions and data storage may be provided.

The patient data model 240 is formed by the data layer. This bottom foundational layer represents the health data for a patient. This layer shows the variety of in situ data sources and clinical data to inform the digital twin patient model. The patient data model includes patient-specific health data from various clinical sources 244, such as electronic health records (EHR), picture archiving and communications system (PACS), laboratory information systems (LIS), global PACS (GPACS), pathology system, radiology information system (RIS), or other source of clinical data provided by physicians, hospitals, or other medical groups. The patient data model 240 includes patient-specific health data from self-reporting 242, such as journaling or application solicited input of symptoms, health goals, appointments, and/or medical schedules. The patient data model 240 includes patient-specific health data from wearables 246, such as from Apple Healthkit, FitBit, Google Fit, and/or other health applications available to patients. The patient data model includes patient-specific health data from applications or input of physical appearance 248, such as a photograph, height, weight, and/or muscle mass. Additional, different, or fewer sources may be provided. Many of the sources are patient-generated, such as self-reported sources 242, wearables 246, and/or other input (e.g., from a camera for photo of physical appearance 248). Either through sensors on consumer products, such as mobile devices or watches, or through patient entry directly to the patient data model 240 or into an application for patient tracking, the patient provides information to be used as PGHD.

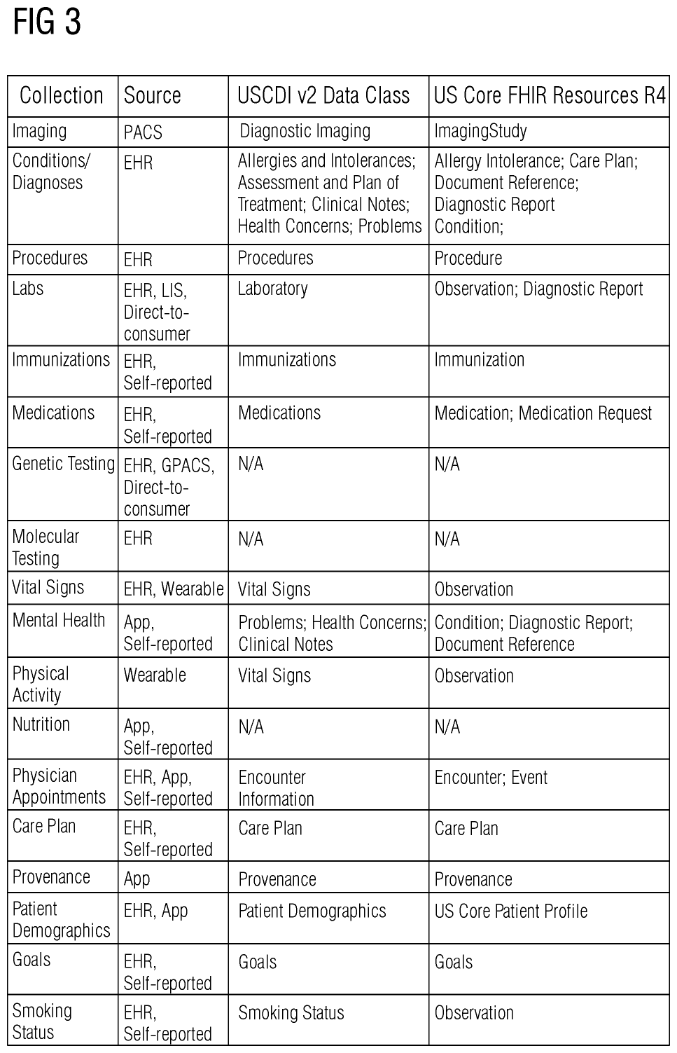

In one embodiment, the patient data model 240 includes patient-specific health data from various sources such as EHR, PACS, LIS and PGHD (wearables, and self-reported data). FIG. 3 shows a list of data categories in a comprehensive patient data model 240 along with the source and two possible formats of the data. The computer populating the patient data model 240 establishes connectivity to wearables and digital health mobile applications via their respective application program interfaces (APIs), to clinical servers (e.g., EHR) by exchanging data objects through fast healthcare interoperability resources (FHIR) or other standards, to PACS and other medical data servers (e.g., LIS) with site-specific protocols, and to the patient or physician by creating interfaces 222 for the user to input self-reported outcomes. Once the connections to various servers and data sources are established, the patient data model 240 is populated. The data model 240 complies with US regulations (e.g., 21s Century Cures Act, Information Blocking Rules, and/or ONC Rules for Interoperability) that provide guidance on the types of data elements (USCDI v2) and their FHIR representation. Healthcare IT systems provide the minimum data elements in USCDI via FHIR APIs and hence the framework accommodates any changes in healthcare data landscape. This also enables the application to fetch the required data from EHRs and to export them back in a seamless manner for use in the clinical workflow.

The health data in the patient data model 240 is updated by replacing, removing, and/or adding health data. For example, the computer implementing the patient data model 240 periodically pulls information from the sources. As another example, one or more sources push information to the patient data model 240. In one embodiment, the flow of data to the patient data model 240 is regularly updated with connectivity to wearables, point of care devices, at-home lab tests, direct-to-consumer genetic testing, or other sources. The frequency of update may be different for different sources.

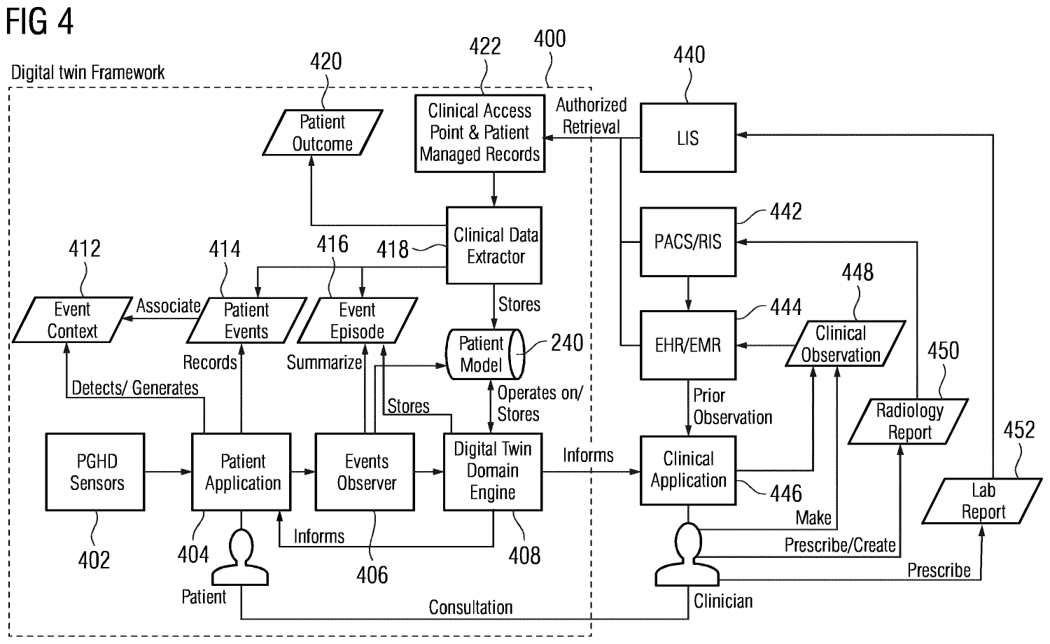

FIG. 4 shows the framework 400 as implemented by software to create and use the patient data model 240. The data flow and transformations for creating the patient data model 240 are represented. The digital twin framework 400 imports data from clinical systems (e.g., LIS 440, PACS or radiology system (RS) 442, and/or EHR or electronic medical records (EMR) 444. The clinical data may be provided through, gathered by, or processed by a clinical application 446, such as provided to a physician or medical practice for use of health data for a patient from the various clinical systems. The clinical application 446 provides an interface for clinical observations 448 by medical professionals for entry into the EHR/EMR 444. The clinician may also prescribe or create a radiology report 450 for inclusion in the PACS/RIS 442 and/or prescribe tests resulting in a laboratory report 452 included in the LIS 440.

Referring again to FIG. 1, the patient data model is populated in act 100 by formatting clinical data from the healthcare systems (e.g., LIS 440, PACS 442, and/or EHR 444 into a format for the patient data model 240. As represented in FIG. 4, the clinical data for a patient is accessed 422 using privacy protective communication standards. The clinical data extractor 418 formats the clinical data into a format used by the patient data model 240. Some data may be combined, discarded, or processed by the clinical data extractor 418 as part of formatting the clinical data into the field definitions or other format of the patient data model 240. In one embodiment, the fields for the patient data model 240 for clinical data follow a same standard as used by the clinical systems, so the formatting may not change the format but may perform some selection or winnowing. The extractor 418 may determine patient outcomes 420 from previous treatment reflected in the clinical data.

Acts 102-106 are for one embodiment for populating the patient data model 240. Additional, different, or fewer acts may be provided, such as acts for accessing, selecting, and/or configuring for data mining or import.

In act 102 of FIG. 1, the computer formats clinical data for a patient. The clinical data is accessed from one or more sources of medical healthcare, such as a physician, medical practice, hospital, imaging center, healthcare facility, and/or other healthcare entity. The sources are databases, such as LIS 440, PACS or RIS 442, and/or HER 444. A clinical application 446 may be accessed to gather data and/or as a tunnel through which data is collected.

The computer formats the clinical data. The format may be by translation, selection, rewriting to different codes, text, or fields, and/or conversion. The format of data for the same or different fields of the patient data model 240 may be different than the format of the clinical data as accessed. The clinical data 240 is formatted for conversion into the patient data model 240. In one embodiment, the formatting is selecting where the patient data model 240 otherwise uses the same format as the databases used to store the clinical data.

In the embodiment of FIG. 4, the interface between the clinician and clinical functional units such as PACS/RIS 442, EHR/EMR 444, LIS 440 etc. clinical systems are guided by medical archive system standards and tend to evolve very slowly in practice. These clinical systems' content is usually in the form of summary reports and actionable recommendations, which may be imported to the patient data model 240 through the clinical access point 422 using the clinical data extractor 418. FIG. 4 shows each major functional unit (rectangular blocks) of the envisioned data flow and their associated data (parallelogram blocks). The formatting by the clinical data extractor 418 follows a standard for medical data sharing.

In act 104 of FIG. 1, the computer formats PGHD from the patient into a format for the patient data model 240 of the framework system. The PGHD is from one or more wearable sensors, health applications, patient input, camera, and/or monitors. The PGHD is accessed through a connection to an application programming interface of a patient device of a patient, such as a computer, mobile phone, and/or wearable sensor. The access may be to a server that collects the PGHD.

In one embodiment, the PGHD is from an application. The patient enters information into the application, such as the self-reported data 242 (e.g., symptoms, health goals, appointments, and/or medication schedules) and/or as the physical data 248 (e.g., height, weight, and/or muscle mass). The application or data from the application is accessed.

In another embodiment, the PGHD is from a wearable, such as wearable data 246. The wearable includes a sensor, such as a heart rate, pressure, step frequency, activity level, or temperature sensor. Other e-health sensors may include breathing cycle, oxygen saturation, or glucose level sensors. More than one sensor may be provided, such as two or more sensors in a same housing or different housings.

The sensor is wearable. For example, the sensor is part of a watch or strap on device for every-day use or use by the patient outside of a medical facility. The sensor may be part of a necklace, watch, strap, clothing, or pack for being worn on the wrist, neck, angle, arm, leg, back, waist, or other part of the body of the patient while outside the healthcare facility. In alternative embodiments, the sensor is not mobile, but is available to the patient at home or outside of a medical facility for regular sensing.

The sensor performs readings from the patient. The sensor acquires signals representing the patient at any time. Based on a timer or a trigger (e.g., user activation or occurrence of an event), regular, periodic, or on-going readings from the patient are performed. A signal or signals in analog or digital form are acquired at least every hour, but other rates may be provided. Irregular readings may occur.

The format, availability, and/or content of PGHD (e.g., from sensors and patient facing applications) may change rapidly with the pace of consumer devices. In situ patient data are often collected as large sets of unfiltered event samples over time, unsuitable for clinical actions without further analytics. The formatting of act 104 alters the PGHD into a different format for the patient data model 240, such as by reduction or alteration. The data is reformatted for the patient data model 240.

In one embodiment, the PGHD is acquired by the wearable at a rate more frequent than desired by the patient data model 240. Similarly, the PGHD from an application, which has another purpose, may not be in a format for clinical purposes. The formatting populates the patient data model 240 with a periodic aggregate or summary, reducing the frequency of the data and/or altering the content for clinical purposes. For example, the wearable generates heart rate data continuously or periodically at least once a minute. A physician may be overwhelmed reviewing data representing a long period of time. The formatting instead summarizes over a greater period, such as providing a daily summary of the heart rate. The application programming interface of the wearable application is accessed to gather the data, which is then formatted by summarization.

The PGHD is compressed. The compression may be using digital compression and/or by summarization. For example, an average heart rate and variance of the heart rate over 24 hours is determined as a summary, compressing data collected each second to less frequent data for a day. As another example, event episodes are identified. The compression is a reduction in data where most or all of the data representing normal is discarded but data associated with an event or episode of non-normal is maintained or provided with more information.

FIG. 4 shows one embodiment where event context is used with the event data to compress the data from the wearable sensor 402 to periodic event episodes 416. The events observer function 406 analyzes event sample collections 414, together with their associated event contexts 412 to produce periodic digested patient event episodes 416 before processing by a digital twin engine 408 and storing into the patient data model 240. An event context 412 contains environmental data associated to the event sample, such as the activity that the patient is doing. The event context 412 may be an overall context of the patient indicating their health status at the time of the sample collection. The event context 412 is captured by the patient facing application 404 as explicit patient/care giver reported inputs, or implicitly via patient location, appointments, or prescribed activities instructed within the application 404. Other sources may provide the context, such as information from a different application where the information represents a same or similar time as an event. The events observer 406 may perform local analytics about the collected event samples 414, such as identifying patterns. The local analytics provides for the event episode 416, which may range from simple aggregative measures such as computing simple statistics (average, mean, max, variance etc.) over a regular time range (day/time of the week/day), to more advanced pattern observations such as correlation between sample values and contextual events (e.g., sample peak value after start of a prescribed activity). The event observer 406 normalizes PGHD to contextualized patient episodes statistically, while both reducing data size and dimensionality, as well as filtering out anomaly events, and variability between usage, device and application context. This formats the PGHD as event episodes 416 for the patient data model 240, including compressing the PGHD to a reduced volume or amount of data.

In act 106 of FIG. 1, the clinical data and the PGHD as formatted for the patient data model 240 are integrated into the patient data model 240. The fields of the patient data module 240 are populated with the data in the format or formats defined for the types of data. The common framework has fields, labeling, memory sections, relationships, and/or other data arrangements relating the different types of data in the one scheme. Both the clinical data and the PGHD are integrated together as the patient data model 240.

In act 110, the computer provides an interface or interfaces to the patient data model 240. The interface provides for access to the clinical data and the PGHD. The access is provided for the patient and/or the physician. The same or different interfaces are provided for the patient and the physician, such as one or more interfaces for the physician and one or more different interfaces for the patient.

FIG. 2 shows example interfaces 220 of the framework. The user interface layer represents the user interfaces to various operators contributing to and interacting with the data flow within the digital twin framework. In the example of FIG. 2, a patient journal user interface 222 is provided. This journal is separate from but may integrate or be replaced by any user interface for third party applications for the patient. The journal user interface 222 provides for viewing and/or entering self-reported health information 242. An imaging navigation and DICOM view user interface 224 allows for patient and/or physician viewing, selection, or input of medical images or other clinical data, such as the medical data 244 or PACS or RIS information. The medical records screen user interface 226 allows for patient and/or physician viewing, selection, and/or input of non-image clinical data, such as notes and records form the EHR of the medical data 244 and/or formatted (e.g., event episodes 416) PGHD wearables data 246. An activity monitor screen user interface 228 allows for patient and/or physician viewing, selection, or input of activity, such as indicated from the PGHD wearable data 246 (e.g., from event episodes 416). The personalized two or three-dimensional avatar user interface 230 allows for patient viewing, selection, and/or input of data. The avatar user interface 230 may be combined with other interfaces to simplify or assist the patient in navigation. The avatar displayed on the avatar user interface 230 may be generated and/or represent characteristics of the patient, such as using a photo of the patient, height, weight, and/or muscle mass of the appearance data 248.

In one embodiment, the avatar user interface 230 displays an avatar of the patient. The size, shape, color, facial features, animation, and/or another characteristic of the avatar represents the patient. The avatar may incorporate a photo of the patient, such as the body and/or face of the patient. In other approaches, the avatar is an animated graphic representing the patient or representing a generic or selected body. One or more characteristics of the avatar, such as the animation sequence, added graphics, expression, size, shape, marker, and/or another display, represent the current health status of the patient. For example, a graphic for lighting bolts to represent pain are overlaid on the avatar at a region associated with pain occurring for the patient. As another example, the avatar changes to reflect a current health score or another biomarker for the patient, such as a color (e.g., red for poor health and blue for good health).

In one embodiment, the virtual avatar is customized in the likeness of the patient. The virtual avatar is computed based on a depth capture photograph of the patient and/or input parameters provided by the patient such as: height, weight, body type, hair style and color, and accessories. The virtual avatar or the digital twin of the patient is a digital representation of the patient's physical health and wellness. The avatar will show animations to indicate health status and/or physical activity of the patient.

The avatar may be used for interaction with other user interfaces 220. For example, the avatar is used to display a medical image from the clinical data. The patient selects a part (e.g., landmark or anatomical region) of the avatar for which a medical image is to be shown. The medical imaging navigation interface 224 allows the patient to browse through their own medical images. The digital avatar of the patient serves as a navigation system. The patient browses through their medical images through the display of landmarks on anatomical regions of their avatar for which corresponding imaging is available. A DICOM viewer may be adapted to a mobile screen or computer to view a patient's medical imaging records. An alternate patient-friendly view of the medical imaging may be presented with cinematic rendering of the imaging data. Medical images will be presented to the patients in the form of animated views (e.g., change of viewer perspective in rendering) of organs along with visual aids to educate the patient about their anatomy and disease.

The user interfaces 220 may be used to provide a summary of the PGHD to the physician. To simplify the amount of data for review by a busy physician, a summary of the PGHD is presented. In addition to the imaging, a dashboard view is presented for PGHD to represent the overall health data of the patient, including conditions/diagnoses, allergies, current medications, etc. Longitudinal data such as physical activity, lab values, and vital signs may be displayed as graphs that show the change in patient's health status over time. The event episodes or other summary from the PGHD are provided to the physician as well as access to the clinical data.

The patient can set wellness goals using the patient journal interface 222. The goals may be achieving a certain health score or target BMI. The avatar user interface 230, patient journal user interface 222, and/or activity monitor screen user interface 228 may provide visualization of their progress towards this goal overtime. The physician may likewise review progress or results for the goal or goals.

The user interfaces 220 may be used for patient interaction and sharing of data with their healthcare providers (e.g., physicians). For regular consultations, a bidirectional connection is established between EHR 444 and the patient data model 240 so that the patient is able to share their health data with their providers for a consult. A communication channel establishes a telemedicine portal so that patients are able to communicate with their providers. In addition to consultation, the framework also provides the possibility for providers to monitor their patients remotely. For example, the providers can monitor the patients' vitals and health score to evaluate their response to medications or a new treatment.

Referring to FIG. 2, the framework includes an application layer 200. Analytic and integrated diagnostic applications 200 are provided to improve patient engagement and outcomes based on the data in the patient data model 240. Any of various applications may be provided. In the example shown, a predication and modeling application 202, remote patient monitoring application 204, patient-centric application 206, imaging-related application 208, data management application 210 and avatar generation application 212 are provided. Additional, different, or fewer applications 200 may be used.

The prediction and modeling application 202 uses the clinical data and/or PGHD of the patient data model 240 to predict for the patient and/or to model the patient. Due to the integrated data, the framework and corresponding patient data model 240 may be used by the prediction and modeling application 202.

In one embodiment, a digital twin domain engine 408 (see FIG. 4) takes the episode event data 416 as input for artificial intelligence (AI)-driven analysis and simulations. The episode event data 416 may include information from PGHD and clinical data. Alternatively, the episode event data 416 from PGHD and clinical data are used by the AI. A different AI may be provided for different domains, such as one for cardiac and one for digestive.

One or more patient clinical access points 422 may be used to broker access to specific clinical data not in the patient data model 240. The access point 422 provides access meeting various data privacy compliance and allow the patient to manage their data. Standards such as SMART on FHIR or other protocols may facilitate access interoperability. Consolidated profiles and observations about the patient over time is stored in the common patient model representing a digital twin based on the collected patient data from which specific trained models (AI as a machine-learned model) can be derived from to gain patient specific insights.

In act 120 of FIG. 1, the computer generates a biomarker for the patient. The biomarker is a heath score, risk, indicator, or value representing the health of the patient. For example, a health score represents an overall health of the patient. The data available in the patient data model 240 is used to compute an overall health score of the patient. The health score will be updated with the addition of new information that integrates daily PGHD from wearables and self-reported to surveys with the medical data of the patient obtained from EHR systems. The health score provides an evolving view of the health status of the patient. As another example, the data from the patient model is used to predict risk scores for most common chronic conditions based on changes in daily activity. In other examples, a disease specific biomarker (e.g., current heart health or risk of heart attach), a biomarker indicating a change leading to an adverse event, a biomarker indicating a response to treatment and/or medication, and/or other biomarkers are generated.

The biomarker is generated by a machine-learned model implemented as a domain engine 408. The machine-learned model generates a value for the biomarker in response to input of PGHD and/or clinical data from the patient data model 240 and/or other sources. The machine-learned model may define the types of information to be input, such as inputting only some or relevant data of the patient data model 240.

Acts 122 and 124 represent one embodiment for generating the biomarker. Additional, different, or fewer acts may be provided.

In act 122, clinical data for the patient is accessed from a healthcare facility or a database managed by a healthcare facility. The access is direct or indirect. For indirect, the access may be to the patient data model 240, where the patient data model 240 was previously populated with the clinical data. The data to be accessed is defined by the machine-learned model to be applied.

In act 124, the AI analyzes the health of the patient based on the clinical data and the event episodes (i.e., periodic aggregate or summary of PGHD). Data relevant for predicting the biomarker is identified and input to the machine-learned model. The machine-learned model outputs a value for the biomarker in response to the input. The value may represent the patient without reference to time or may represent the health at a given time.

In act 130, the computer, using a display, outputs one or more health scores or biomarkers. The output is on the appropriate user interface 220. In one example, the health to the patient is presented on a user interface 220 including an avatar of the patient. A characteristic of the avatar represents the health or biomarker. Alternatively, or additionally, the output is to memory storing the patient data model 240. The values of the biomarkers are added to the patient data model 240.

To clinically use the machine generated insights gained from any digital twin engine 408, a clinician may evaluate such insights via domain specific clinical applications 446. Together with any additional consultation information outside of the digital software, the clinician ultimately approves or creates the final clinical recommendations in the form of reports and summary compatible with clinical practices and existing clinical archive systems and workflows, thus performing the final data transformation of PGHD into standard records used in clinical practice.

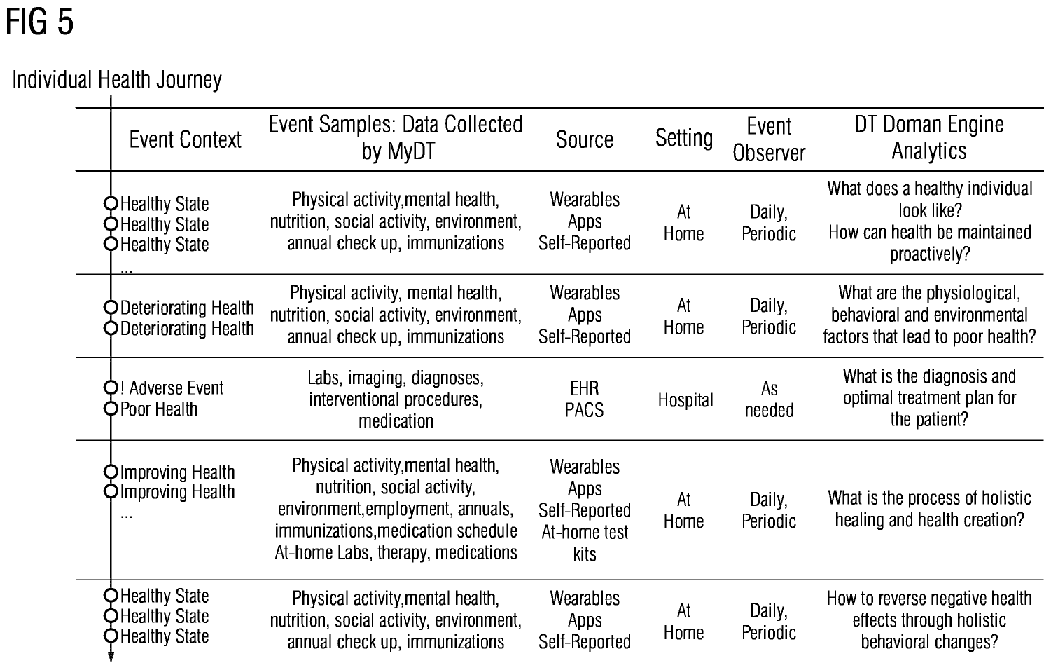

FIG. 5 illustrates an example use of the framework and corresponding patient data model 240. A patient's health journey along with touch points with the framework is represented. On the left, an arrow indicates various timepoints in a patient's health journey. The next column indicates the event context 412 indicating their health status. The health score is used as context for the period. The next three columns show the event samples (PGHD and/or clinical data) collected corresponding to the timepoint in the health journey, source of the samples, and location or setting where the samples are collected. The next column shows the frequency of the event observer 406 that collects these event samples, consolidates them, and stores them as event episodes 416. The last column shows the analytics performed by the domain engine 408 at each timepoint. The AI provides answers or information that may be used to answer one or more questions.

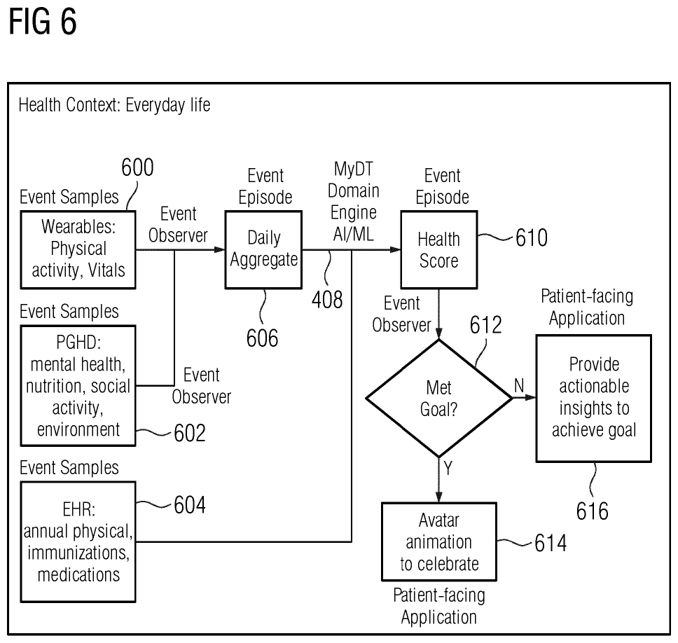

For healthy individuals, the touchpoints are with wearables and self-reported data. The framework can provide insights on how health can be proactively maintained by managing physical and mental health proactively by monitoring the progress of their health scores, as seen in FIG. 6. In this context, the even samples are collected from PGHD 600, 602 and consolidated into daily aggregates 606 of event episodes 416. The domain engine 408 computes a health score 610 based on these daily aggregates 606, along with the clinical context obtained from EHR event samples 604, resulting in a health score 610 of the patient being computed every day. This health score 610 is stored as an event episode 416 or context 412. A secondary event observer 406 monitors the health score 610 daily to check 612 if the score 610 meets the goals that the patient has set for themselves. When the individual meets their goals, a celebratory animation 614 of the avatar is displayed. For individuals yet to meet their goal, actionable insights 616 are provided to reach their goal.

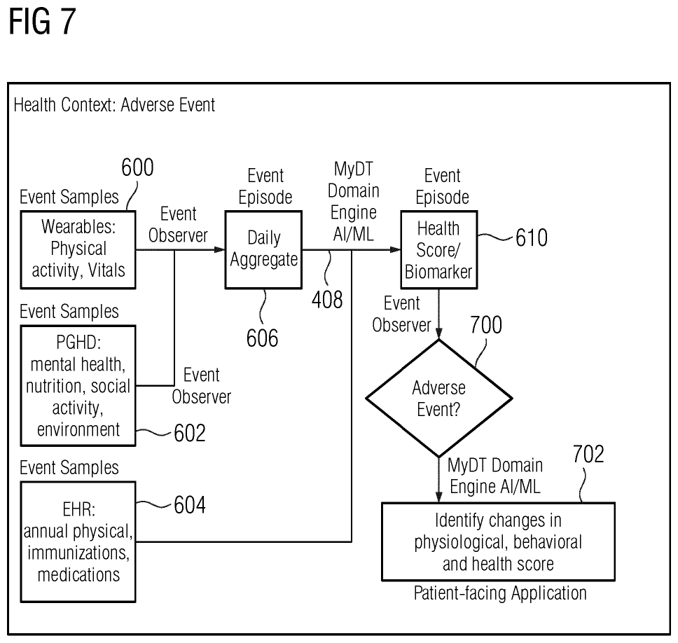

FIG. 7 shows use of the framework for observing adverse events. In case of an adverse event, longitudinal data 600, 602 that is collected from the patients through wearables and other PGHD, along with the context of their medical history is analyzed. In this context, an event observer 700 monitors the health score or other biomarker 610 for the patient daily. If an adverse event is detected, the domain engine uses a machine-learned model to identify physiological, behavioral and environmental changes 702 leading to the adverse event.

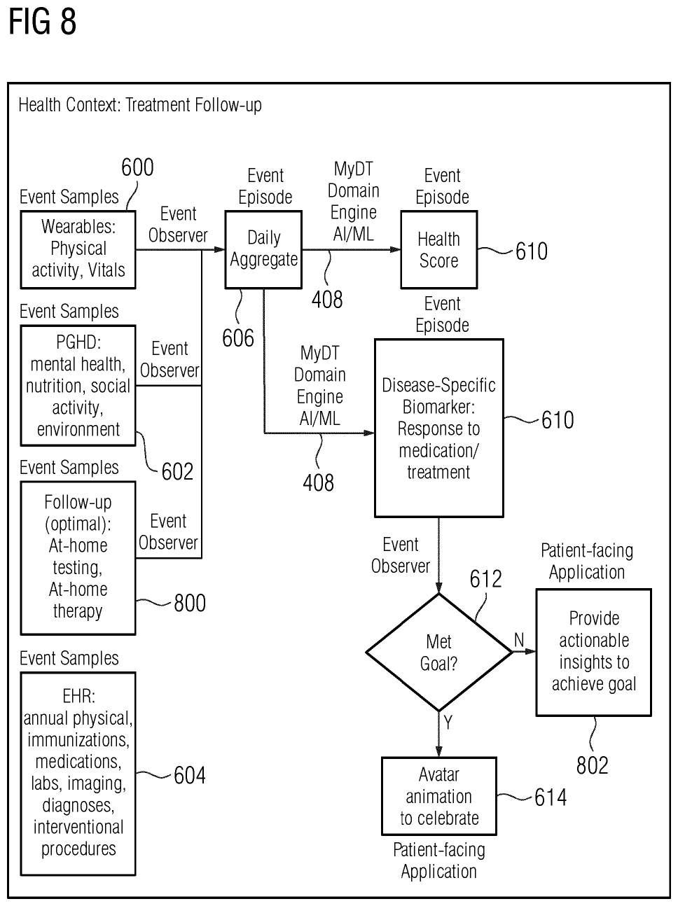

When the patient is at a hospital or a clinic for diagnosis and treatment of their condition, the framework can be used to share medical and behavioral history data with their providers to enable personalized diagnosis and treatment decision making. The framework may be helpful for individuals who have undergone or are undergoing a treatment and medication regimen, as seen in FIG. 8. In this context, additional event samples 800 may be collected from at-home lab tests or therapies as dictated by the treatment regimen. In addition, there might be additional EHR data 604 collected from the clinical data extractor 418 during the course of their diagnosis and treatment. The domain engine 408 computes a disease-specific biomarker 610 to analyze the patient's response to treatment or medication. An event observer 612 checks to see if the patient is responding to the treatment or medication. A machine-learned model in the domain engine 408 analyzes the longitudinal health score 610 and disease-specific biomarker 610 to monitor the health progress of the individual and evaluate response to therapy. Reaching a goal may be celebrated 614, such as by animation with an avatar. Failure to reach a goal may result in presentation of actionable insights 802 to achieve the goal.

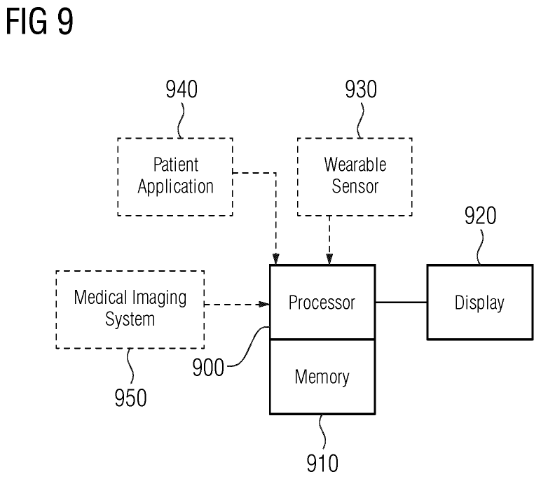

FIG. 9 shows one embodiment of a system for modeling patient data in a framework. The system includes a processor 900, memory 910, and display 920. Additional, different, or fewer devices may be provided, such as a wearable sensor 930, mobile device with a patient application 940, and/or a medical imaging system 950. While one processor 900 and memory 910 is provided, multiple processors and memories may be used, such as a server for clinical data sources and the processor 900 of the patient computer or another server for populating and/or using the patient data model 240.

The computing components, devices, or machines of the system, such as the medical imaging system 950, patient application 940, and/or the processor 900 are configured by hardware, software, firmware, and/or design to perform the acts of the method of FIG. 1 or other acts. The computing components operate independently or in conjunction with each other to perform any given act. The acts are performed by one of the computer components, another of the computing components, or a combination of the computing components. Other components may be used or controlled by the computing components to scan, sense, display, measure, analyze, or perform other functions.

The medical imaging system 950 is any now known or later developed modality for scanning a patient. The medical imaging system 950 scans the patient for an anatomical region. For example, a C-arm x-ray system (e.g., DynaCT from Siemens), CT like system, or CT system is used. Other modalities include MR, x-ray, angiography, fluoroscopy, PET, SPECT, or ultrasound. The medical imaging system 950 is configured to acquire the medical imaging data representing the patient. The medical imaging system 950 acquires imaging clinical data.

The wearable sensor 930 is a consumer product, such as a wearable watch, or is a medical monitor for use outside of a healthcare facility. The wearable sensor 930 is a heart rate sensor, temperature sensor, EKG sensor, pulse-oximetry sensor, breathing sensor, glucose sensor, step or activity sensor, a pressure cuff, another sensor, or combinations thereof. Electrodes, accelerometers, gyroscopes, infra-red sensor, or other sensing devices may be used. The wearable sensor 930 provides PGHD.

The wearable sensor 930 is configured by hardware, software, and/or firmware to regularly sense the patient. Any period may be used for on-going sensing, such as periodically sensing every number of seconds, minutes, or hours. Daily or weekly sensing may be used in other embodiments. For real-time or continuous sensing, the sensing is every minute or more frequent.

The patient application 940 is an application on a mobile device or other computer for interaction with and/or health monitoring of the patient. The patient application 940 collects information about the patient, such as being a health journal to set goals and receive patient-provided input relative to those goals.

The memory 910 is a buffer, cache, RAM, removable media, hard drive, magnetic, optical, database, or other now known or later developed memory. The memory 910 is a single device or group of two or more devices. The memory 910 is part of a computer with the processor 900, within a cellular phone (e.g., smart phone), or is outside or remote from other components. The memory 910 is configured (e.g., formatted or indexed for data storage) by the processor 900 or another device.

The memory 910 is configured to store the patient data model 240. The memory 910 stores clinical data from a database of a healthcare facility and stores PGHD from the wearable sensor 930 and/or application 940 on a patient device. The clinical data and the PGHD are stored in the framework common to both the clinical data and PGHD.

The memory 910 may store values from the on-going sensing of the wearable sensor 930. The values from the wearable sensor 930 are received and stored for use in generating event episodes and/or context to be included in the patient data model. A summary or other compression of PGHD may be generated by the processor 900 and stored in the memory 910.

The memory 910 is additionally or alternatively a non-transitory computer readable storage medium with processing instructions. The memory 910 stores data representing instructions executable by the programmed processor 900. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 900 is a general processor, digital signal processor, three-dimensional data processor, graphics processing unit, application specific integrated circuit, field programmable gate array, digital circuit, analog circuit, tensor processor, AI processor, combinations thereof, or other now known or later developed device for managing stored data, user interfaces, and/or applications of the framework. The processor 900 is a single device, a plurality of devices, or a network. For more than one device, parallel or sequential division of processing may be used. Different devices making up the processor 900 may perform different functions, such as populating and operating the patient data model by one processor and presenting the user interfaces and/or applications by one or more other processors. The processor 900 operates pursuant to stored instructions to perform various acts described herein.

The processor 900 is configured to generate a user interface for interacting with the framework by a patient. The user interface for the patient includes access to the clinical data. The user interface may include any application, such as a prediction application. The prediction application is configured to generate a health score for the patient based on both the clinical data and the PGHD from the framework.

The processor 900 may curate the data, such as formatting the clinical data and/or the PGHD for the patient data model. For example, the processor 900 is configured to summarize the PGHD. The processor 900 may be configured to generate a user interface for interacting with the framework by a physician. The summarization is available to the physician in the user interface, allowing for rapid review without requiring data analysis based on raw data.

In one embodiment, the processor 900 is configured to generate the user interface with an avatar of the patient. Using the user interface, the processor 900 may display information from the clinical data based on selection of part of the avatar.

The display 920 is a CRT, LCD, plasma, projector, printer, or other output device for showing an image. The display 920 is configured by a display plane buffer to display the display part of the user interface. Text, graphs, charts, images, values of biomarkers, event episodes, actionable insights, avatar, animations, clinical data, PGHD, and/or other information may be displayed.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for integration of health information into a framework system, the method comprising:
formatting clinical data for a patient from a first source of medical healthcare into a first format for a patient data model of the framework system;
formatting patient-generated health data from the patient into a second format for the patient data model of the framework system;
integrating the clinical data and patient-generated health data as formatted into the patient data model for the patient, the integration storing both the clinical data and the patient-generated health data together as the patient data model;
providing an interface or interfaces to the patient data model, including access to the clinical data and the patient-generated health data, for both a patient and a physician;
generating, by a machine-learned model, a biomarker for the patient, the machine-learned model generating in response to input at least some of both of the clinical data and the patient-generated health data from the patient data model; and
outputting the biomarker.

2. The method of claim 1 wherein formatting the clinical data comprises formatting the clinical data from the first source comprising a picture archiving and communications system (PACS), radiology information system, electronic health record, and/or laboratory information system for a medical practice or facility.

3. The method of claim 1 wherein formatting the clinical data comprises formatting pursuant to a standard for medical data sharing.

4. The method of claim 1 wherein formatting the patient-generated health data comprises formatting where the patient-generated health data is from a wearable sensor.

5. The method of claim 4 wherein formatting comprises compressing data from the wearable sensor provided at a first frequency to a less frequent representation.

6. The method of claim 4 wherein formatting comprises compressing data from the wearable sensor to periodic event episodes based on event context of the data from the wearable sensor.

7. The method of claim 1 wherein formatting the patient-generated health data comprises reformatting data from an application for gathering information input by the patient.

8. The method of claim 7 wherein formatting the patient-generated health data further comprises compressing data from a wearable sensor, and wherein formatting the clinical data comprises formatting the clinical data from the first source comprising a picture archiving and communications system (PACS) or radiology information system, an electronic health record, and laboratory information system for a medical practice or facility.

9. The method of claim 1 wherein providing the interface comprises displaying an avatar of the patient with a characteristic of the avatar representing the biomarker.

10. The method of claim 9 wherein providing the interface comprises displaying the avatar and a medical image from the clinical data, the medical image selected for the displaying based on selection of a landmark or anatomical region of the avatar.

11. The method of claim 1 wherein generating the biomarker comprises generating a health score for overall health of the patient.

12. The method of claim 1 wherein generating the biomarker comprises generating a disease specific biomarker.

13. The method of claim 1 wherein generating the biomarker comprises generating the biomarker as a change leading to an adverse event.

14. The method of claim 1 wherein generating the biomarker comprises generating the biomarker as a response to treatment and/or medication.

15. The method of claim 1 wherein providing comprises providing a summary of the patient-generated health data to the physician.

16. A system for modeling patient data in a framework, the system comprising:
a memory configured to store clinical data from a database of a healthcare facility and to store patient-generated health data from a wearable sensor and/or application on a patient device, the clinical data and the patient-generated health data stored in the framework common to both the clinical data and patient-generated health data;
a processor configured to generate a user interface for interacting with the framework by a patient including access to the clinical data; and
a display configured to display part the user interface.

17. The system of claim 16 wherein the user interface includes a prediction application, the prediction application configured to generate a health score for the patient based on both the clinical data and the patient-generated health data from the framework.

18. The system of claim 16 wherein the processor is configured to summarize the patient-generated health data, and wherein the processor is configured to generate the user interface for interacting with the framework by a physician, the summarization available to the physician in the user interface.

19. The system of claim 16 wherein the user interface includes an avatar of the patient and information from the clinical data based on selection of part of the avatar.

20. A method for integration of health information into a framework system, the method comprising:
populating a patient data model of the framework system through a connection to an application programming interface of a patient device of a patient, the patient data model populated with a periodic aggregate summary of more frequent data collected by an application of the application programming interface;
accessing clinical data for the patient from a healthcare facility;
analyzing, by artificial intelligence, health of the patient based on the clinical data and the periodic aggregate summary;
presenting the health to the patient on a user interface including an avatar of the patient, the avatar having a characteristic representing the health.
